(19) United States
(12) Patent Application Publication
(10) Pub. No.: US 2024/0000170 A1
(43) Pub. Date: Jan. 4, 2024

(54) GLOVE AND ACTION DETERMINATION SYSTEM

(71) Applicant: SINTOKOGIO, LTD., Nagoya-shi (JP)

(72) Inventors: Yoshikane TANAAMI, Nagoya-shi (JP); Miyuki HAYASHI, Nagoya-shi (JP)

(73) Assignee: SINTOKOGIO, LTD., Nagoya-shi (JP)

(21) Appl. No.: 18/325,292

(22) Filed: May 30, 2023

(30) Foreign Application Priority Data
Jun. 30, 2022 (JP) 2022-106270

(51) Int. Cl. A41D 19/00; G01L 1/14; G01P 15/18; A41D 1/00; A43B 5/00; A43B 3/48; A43B 3/44

(52) U.S. Cl. CPC A41D 19/0027; G01L 1/142; G01P 15/18; A41D 1/002; A43B 5/00; A43B 3/48; A43B 3/44; A41D 2500/20; A41D 2600/10

(57) ABSTRACT

A glove includes a capacitive sensor, and conductive threads are woven into a fabric of the glove, the conductive threads serving as the capacitive sensor configured to detect, on the basis of a change in capacitance between the conductive threads adjacent to each other, a change in pressure applied to an area into which the conductive threads are woven.

This Nonprovisional application claims priority under 35 U.S.C. § 119 on Patent Application No. 2022-106270 filed in Japan on Jun. 30, 2022, the entire contents of which are hereby incorporated by reference.

### TECHNICAL FIELD

The present invention relates to a glove and an action determination system.

### BACKGROUND ART

For example, as disclosed in Patent Literature 1, a technique has been known of sewing a conductive woven fabric into cloth such as a glove to achieve a capacitive sensor which detects a change in electric capacity occurring between a human body and the conductive woven fabric.

### CITATION LIST

### Patent Literature

### [Patent Literature 1]

Published Japanese Translation of PCT International Application Tokuhyo No. 2013-534833

### SUMMARY OF INVENTION

### Technical Problem

However, Patent Literature 1 does not disclose the configuration in which a change in pressure is detected on the basis of a change in capacitance caused by increasing proximity between adjacent two of conductive threads which are woven in a fabric.

It is an object of an aspect of the present invention to provide a glove and an action determination system which each have a novel configuration and can detect a change in pressure.

### Solution to Problem

In order to solve the foregoing problem, a glove in accordance with an aspect of the present invention includes a capacitive sensor. Into a fabric of the glove, conductive threads are woven. The conductive threads serve as a sensor configured to detect, on the basis of a change in a capacitance between the conductive threads adjacent to each other, a change in a pressure applied to an area into which the conductive threads are woven.

Further, in order to solve the foregoing problem, an action determination system in accordance with an aspect of the present invention includes a glove, an acceleration sensor, and a controller. The glove includes a capacitive sensor. The acceleration sensor is a sensor configured to detect a movement of an upper limb of a user and is worn around the upper limb of the user. The controller executes a determination process of determining an action of the upper limb of the user. The determination process is executed on the basis of a change in pressure detected by the sensor of the glove and a change in an acceleration of the upper limb of the user detected by the acceleration sensor.

### Advantageous Effects of Invention

According to an aspect of the present invention, it is possible to provide a glove and an action determination system which each have a novel configuration and can detect a change in pressure.

### BRIEF DESCRIPTION OF DRAWINGS

FIG. 1 is a view schematically illustrating a configuration of a glove in accordance with Embodiment 1 of the present disclosure.

FIG. 2 is a block diagram illustrating a configuration of an action determination system in accordance with Embodiment 2 of the present disclosure.

FIG. 3 is a view schematically illustrating the action determination system illustrated in FIG. 2.

FIG. 4 is a block diagram illustrating a configuration of an action determination system in accordance with Embodiment 3 of the present disclosure.

FIG. 5 is a view schematically illustrating the action determination system illustrated in FIG. 4.

### DESCRIPTION OF EMBODIMENTS

### Outlines of Embodiments of the Present Disclosure

First, the following will describe outlines of embodiments of the present disclosure.

### (Item 1)

A glove including a capacitive sensor, wherein:
conductive threads are woven into a fabric of the glove; and
the conductive threads serve as the capacitive sensor configured to detect, on a basis of a change in capacitance between the conductive threads adjacent to each other, a change in pressure applied to an area into which the conductive threads are woven.

### (Item 2)

The glove according to Item 1, wherein the conductive threads are woven into a position corresponding to a portion to which a pressure is to be applied in a hand of a user and which is in an area in contact with at least one selected from the group consisting of: a finger of the user, a palm of the user, and a dorsal surface of the hand of the user.

### (Item 3)

The glove according to Item 1 or 2, further including a transmitting section configured to transmit, to an external device, data indicating a change in pressure detected by the capacitive sensor,
the transmitting section being detachably attached to the glove.

### (Item 4)

An action determination system including:
the glove according to any one of Items 1 to 3;
an acceleration sensor worn around an upper limb of a user and configured to detect a movement of the upper limb of the user; and
a controller configured to execute a determination process of determining an action of the upper limb of the user on a basis of a change in pressure detected by the capacitive sensor of the glove and a change in acceleration of the upper limb of the user detected by the acceleration sensor.

### (Item 5)

The action determination system according to Item 4, further including an external force measurement section configured to measure an external force applied from a lower limb of the user,
the controller being configured to further determine, in the determination process, an action of a whole body of the user on a basis of the action of the upper limb of the user determined and an external force measured by the external force measurement section.

### (Item 6)

The action determination system according to Item 5, wherein the external force measurement section is a force plate configured to measure a load applied from the user.

### (Item 7)

The action determination system according to Item 5, wherein the external force measurement section is footwear including a pressure sensor configured to measure a pressure applied from the user.

### Examples of Embodiments of the Present Disclosure

The following will describe embodiments of the present disclosure in detail with reference to the drawings. Note that, in the descriptions of the drawings, the same components are assigned the same reference sign, and the same descriptions are omitted. In the following embodiments, a boxing game is taken as an example.

### Embodiment 1

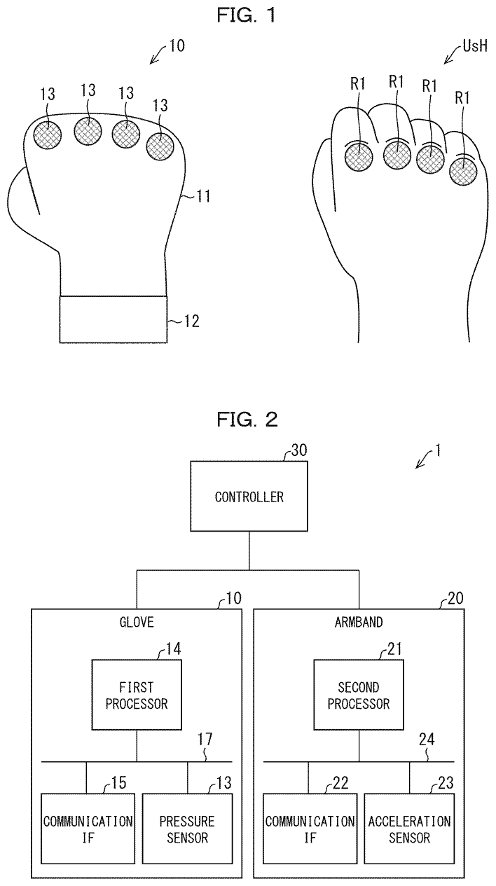

The following will describe a configuration of a glove in accordance with Embodiment 1 of the present disclosure. FIG. 1 is a view schematically illustrating a configuration of a glove 10.

The glove 10 is a glove configured to detect a movement of a hand UsH of a user Us. In an example illustrated in FIG. 1, the glove 10 is a boxing glove. The glove 10 performs wireless communications with an external device. Note that the glove 10 may perform wired communications with an external device.

The glove 10 is constituted by a glove main body 11 and a transmitting section 12. Into a fabric of the glove main body 11, conductive threads are woven. Application of a pressure to an area into which the conductive threads are woven causes a change in a capacitance between the conductive threads adjacent to each other. That is, the conductive threads serve as a pressure sensor 13.

More specifically, the pressure sensor 13 is a capacitive sensor. Application of a pressure to the glove main body 11 causes deformation of the glove main body 11. This causes the conductive threads adjacent to each other to approach each other. The approach of the conductive threads adjacent to each other causes a change in a capacitance between the conductive threads. The pressure sensor 13 detects the change in the capacitance to detect a pressure applied to the glove main body 11. The pressure sensor 13 detects a movement of the hand UsH of the user Us.

The conductive threads are woven at a position corresponding to a portion to which a pressure is to be applied in the hand UsH of the user Us and which is in an area in contact with at least one selected from the group consisting of: a finger of the user Us; a palm of the user Us; and a dorsal surface of the hand of the user Us. That is, the pressure sensor 13 is disposed at the position corresponding to the portion to which a pressure is to be applied in the hand UsH of the user Us. In an example illustrated in FIG. 1, when the user Us wearing the glove 10 hits an object, it is assumed that pressures are applied to areas R1 of the hand UsH of the user Us. The areas R1 are located on a dorsal side of the hand UsH and are areas corresponding to respective bases of the fingers of the hand UsH. In the glove 10, the plurality of pressure sensors 13 are disposed at respective positions corresponding to the areas R1 of the hand UsH.

According to the above configuration, in the glove 10, the conductive threads are woven into each of positions corresponding to respective portions to which pressures are to be applied in the hand UsH of the user Us. Thus, at each of the positions, the pressure sensor 13 is disposed. Therefore, it is possible to more specifically detect a change in a pressure of the hand UsH of the user Us. This makes it possible to detect a more realistic movement of the hand UsH of the user Us.

The transmitting section 12 transmits, to an external device, an output signal indicating a change in pressure detected by the pressure sensors 13. The transmitting section 12 has an interface for communicating with the external device. The transmitting section 12 is detachably attached to a wrist side of the glove main body 11.

According to the above configuration, the transmitting section 12 and the glove main body 11 can be separated from each other. Thus, when the glove 10 gets dirty, it is possible to wash only the glove main body 11. This makes it possible to reduce uncomfortableness that the user Us feels when wearing the glove 10 on the hand UsH.

### Embodiment 2

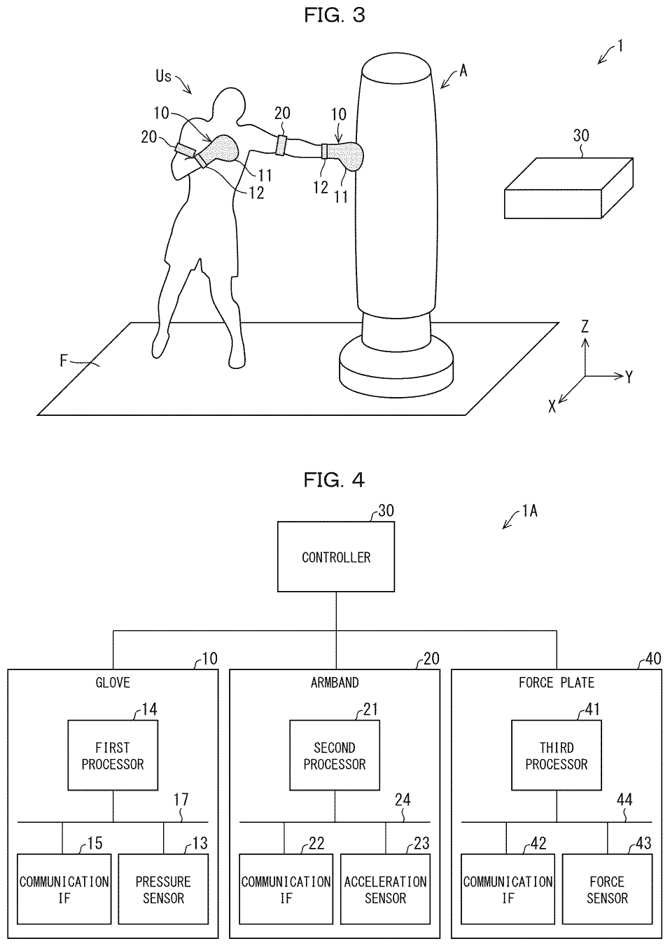

The following will describe another embodiment of the present disclosure with reference to FIGS. 2 and 3. FIG. 2 is a block diagram illustrating a configuration of an action determination system 1. FIG. 3 is a view schematically illustrating the action determination system 1 illustrated in FIG. 2. Embodiment 2 shows an example of an action determination system configured to determine an action of an upper limb of a user Us.

The following description assumes that coordinate axes having three directions, which are an x axis, a y axis, and a z axis, are set as illustrated in FIG. 3. Here, in Embodiment 2, the z axis is an axis parallel to a direction orthogonal to a floor surface F on which the user Us stands, the x axis is an axis orthogonal to the z axis, and the y axis is an axis orthogonal to the x axis and the y axis. The description below assumes that the x axis is a left-right direction with respect to an object A, and the y axis is a front-rear direction with respect to the object A.

Outline of Action Determination System 1

As illustrated in FIGS. 2 and 3, an action determination system 1 includes a glove 10, an armband 20, and a controller 30. The glove 10 and the armband 20 are each configured to be able to perform wireless communications with the controller 30. Note that the glove 10 and the armband 20 may be each configured to perform wired communications with the controller 30.

The glove 10 includes a first processor 14, a communication IF 15, and pressure sensors 13. The first processor 14, the communication IF 15, and the pressure sensors 13 are connected with each other via a bus 17.

The first processor 14 transmits, to the controller 30, an output signal indicating a change in pressure detected by the pressure sensors 13. Examples of the first processor 14 include a central processing unit (CPU) and a graphic processing unit (GPU). The second processor 21 described later is configured in the same manner as the first processor 14.

The communication IF 15 is an interface configured to perform wireless communications with the controller 30. The wireless communications may be performed in accordance with a known standard such as Bluetooth (registered trademark), an infrared ray communication, or a wireless LAN. Note that the communication IF 15 may perform communications with use of a variety of known networks. Alternatively, the communication IF 15 may perform wired communications with the controller 30. A communication IF 22 described later is configured in the same manner as the communication IF 15.

<Armband>

The armband 20 is worn around an upper limb of the user Us and detects a movement of the upper limb of the user Us. The armband 20 includes a second processor 21, a communication IF 22, and an acceleration sensor 23. The second processor 21, the communication IF 22, and the acceleration sensor 23 are connected with each other via a bus 24.

The acceleration sensor 23 is a sensor configured to detect accelerations in the three axial directions of the x axis, the y axis, and the z axis. The movement of the upper limb of the user Us is detected on the basis of an acceleration detected by the acceleration sensor 23. The second processor 21 transmits an output signal indicating a change in acceleration detected by the acceleration sensor 23 to the controller 30 via the communication IF 22.

<Controller>

The controller 30 is a device configured to receive an output signal from the pressure sensor 13 of the glove 10 and an output signal from the acceleration sensor 23 of the armband 20 and comprehensively determine an action of the upper limb of the user Us on the basis of the output signals.

The controller 30 is implemented by, for example, a personal computer (PC) or a programmable logic controller (PLC). The controller 30 includes an interface configured to communicate with, for example, a processor such as a CPU or a GPU, a memory such as a read only memory (ROM) or a RAM (random access memory), and other devices.

[Flow of Operation of Action Determination System 1]

The controller 30 comprehensively determines an action of the upper limb of the user Us on the basis of: a change in acceleration detected by the acceleration sensor 23 of the armband 20; and a change in pressure detected by the pressure sensors 13 of the glove 10. More specifically, the controller 30 determines a kind of the action (such as a bend of the upper limb) of an upper body of the user Us on the basis of a change in acceleration detected by the acceleration sensor 23. The controller 30 determines a kind of the action of the upper limb of the user Us, accuracy of the action of the upper limb of the user Us, and/or a kind of the action of the hand UsH of the user Us on the basis of a change in pressure detected by the pressure sensors 13. In Embodiment 2, the controller 30 determines, through the armband 20, a kind of a punch (such as a straight punch and a hook), which is an action of the upper limb of the user Us. In addition, the controller 30 determines, through the glove 10, accuracy of the punch and a strength of the punch. Note that, herein, the expression “determine the action” includes determinations of (i) a kind of the action, (ii) accuracy of the action, and (iii) a strength of the action.

According to the above configuration, it is possible to more specifically determine the action of the upper limb of the user Us on the basis of a combination of: a change in acceleration of the upper limb detected by the acceleration sensor 23 of the armband 20; and a change in pressure detected by the pressure sensors 13 of the glove 10. This makes it possible to determine a more realistic action of the upper limb.

### Embodiment 3

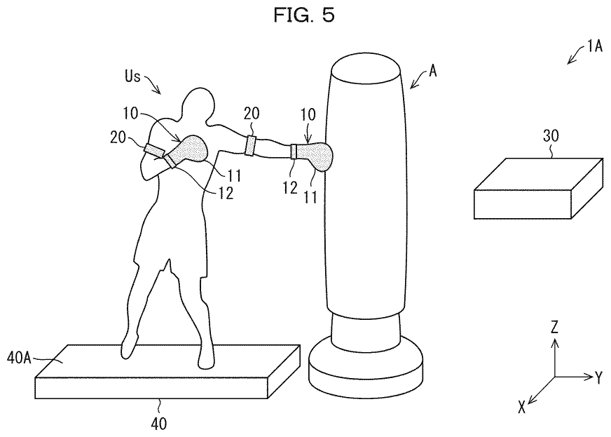

The following will describe another embodiment of the present disclosure with reference to FIGS. 4 and 5. FIG. 4 is a block diagram illustrating a configuration of an action determination system 1A. FIG. 5 is a view schematically illustrating the action determination system 1A illustrated in FIG. 4. The action determination system 1A in accordance with Embodiment 3 further includes, in addition to the configuration of Embodiment 2, a force plate, which is an example of an external force measurement section configured to measure an external force applied from a lower limb of a user Us. Embodiment 3 shows an example of an action determination system configured to determine an action of a whole body of a user.

Here, in Embodiment 3, a z axis is an axis parallel to a direction orthogonal to a surface 40A of a force plate 40 on which the user Us stands, an x axis is an axis orthogonal to the z axis, and a y axis is an axis orthogonal to the x axis and the z axis, as illustrated in FIG. 5. The description below assumes that the x axis is a left-right direction with respect to an object A, and the y axis is a front-rear direction with respect to the object A.

Outline of Action Determination System 1A

As illustrated in FIGS. 4 and 5, the action determination system 1A includes a glove 10, an armband 20, a controller 30, and the force plate 40. The force plate 40 is configured to be able to perform a wireless communication with the controller 30. Note that the force plate 40 may be configured to perform a wired communication with the controller 30.

<Force Plate>

The force plate 40 measures a load, which is an external force applied from the user Us on the force plate 40. As illustrated in FIG. 4, the force plate 40 includes a third processor 41, a communication IF 42, and force sensors 43. The third processor 41, the communication IF 42, and the force sensors 43 are connected with each other via a bus 44. The third processor 41 is configured in the same manner as the first processor 14. The communication IF 42 is configured in the same manner as the communication IF 15.

In Embodiment 3, as the force sensor 43, a six-axis force sensor is used. The force sensors 43 detect: forces Fx, Fy, and Fz respectively in directions of the x axis, the y axis and the z axis which act on the force plate 40; and moments Mx, My, and Mz respectively about the x axis, the y axis, and the z axis which act on the force plate 40.

The force sensor 43 is disposed at each four corners of the force plate 40 which has a rectangular shape. The force sensors 43 measure moments and a load which are applied from the user Us on the force plate 40. The third processor 41 transmits, to the controller 30 via the communication IF 42, an output signal indicating a change in load detected by the force sensors 43 and an output signal indicating a change in moment detected by the force sensors 43.

[Flow of Operation of Action Determination System 1A]

The controller 30 determines an action of a lower limb of the user Us on the basis of a load and moments detected by the force sensors 43 of the force plate 40. The controller calculates the center of a pressure applied to a foot of the user Us on the basis of a load measured by the force sensors 43. As a result, a position of the gravity center of the user Us is detected. In addition, the controller 30 calculates a moment which acts on the foot of the user Us on the basis of the moments detected by the force sensors 43. More specifically, the controller 30 calculates a moment about a direction (direction of the z axis in FIG. 2) orthogonal to the surface 40A of the force plate 40 on which the user Us stands. As a result, a direction of a movement of the foot of the user Us is detected. On the basis of the center of the pressure of the foot calculated and the moment calculated, determined are a kind of the action (such as walking, running, or jumping) of the lower limb of the user Us and strengths (such as a degree of a stepping-in and a jumping ability) of an action of the lower limb of the user Us. In Embodiment 3, the force plate 40 detects a strength of a stepping-in and a form of a lower body at the time when the user Us performs a punch.

The controller 30 determines an action of a whole body of the user Us on the basis of: a change in acceleration detected by an acceleration sensor 23 of an armband 20; a change in pressure detected by pressure sensors 13 of a glove and a change in load detected by the force sensors 43 of the force plate 40. For example, in Embodiment 3, it is possible to detect a form of the whole body and how the whole body exerts a force at the time when the user Us performs a punch.

According to the above configuration, it is possible to comprehensively determine an action of the whole body of the user Us on the basis of a combination of: a change in acceleration detected by the acceleration sensor 23 of the armband 20; a change in pressure detected by the pressure sensors 13 of the glove 10; and a change in load detected by the force sensors 43 of the force plate 40. This makes it possible to determine a more realistic action of a whole body.

### Embodiment 4

The following will describe another embodiment of the present disclosure. The force plate 40 measures an external force applied from a user Us in Embodiment 3 above, but a footwear in which a pressure sensor is disposed may be used instead of the force plate 40. The footwear is an example of an external force measurement section.

The footwear is worn around a lower limb of a user Us. Examples of the footwear include a room boot, a sock, and a shoe. Further, the footwear is not limited to one for indoor use and may be one for outdoor use. In Embodiment 4, a room boot for indoor use is taken as an example.

The room boot measures a pressure, which is an external force applied from a lower limb of the user Us using the room boot. The room boot performs wireless communications with a controller 30. Note that the room boot may perform wired communications with the controller 30. The room boot includes a fourth processor, a communication IF, and a pressure sensor.

The fourth processor, the communication IF, and the pressure sensor are connected with each other by a bus. The pressure sensor is disposed on a bottom portion (sole portion) of the room boot. In the room boot, the pressure sensor is disposed at a position corresponding to a portion to which a pressure is to be applied in a toe and a sole of the foot of the user Us. The pressure sensor measures a pressure applied from the lower limb of the user Us. The fourth processor transmits an output signal from the pressure sensor to the controller 30 via the communication IF.

The controller 30 receives output signals from: an acceleration sensor 23 of an armband 20; pressure sensors 13 of a glove 10; and the pressure sensor of the room boot and comprehensively determines an action of a whole body of the user Us on the basis of the output signals.

The controller 30 determines an action of the whole body of the user Us on the basis of: a change in acceleration detected by the acceleration sensor 23 of the armband 20; a change in pressure detected by the pressure sensors 13 of the glove 10; and the pressure measured by the pressure sensor of the footwear. For example, in Embodiment 4, it is possible to detect a form of the whole body and how the whole body exerts a force at the time when the user Us performs a punch.

According to the above configuration, it is possible to comprehensively determine an action of the whole body of the user Us on the basis of the combination of: a change in acceleration detected by the acceleration sensor 23 of the armband 20; a change in pressure detected by the pressure sensors 13 of the glove 10; and a change in pressure of detected by the pressure sensor of the footwear. This makes it possible to determine a more realistic action of a whole body.

In Embodiment 4, the position of the pressure sensor disposed in the room boot is limited. However, the position of the pressure sensor is not limited to this. In the room boot, the pressure sensor may be disposed at a position corresponding to a dorsal surface portion of a foot of the user Us or a heel portion of the user Us. The position at which the pressure sensor is disposed can be changed as appropriate depending on the intended use of the footwear.

### Another Embodiment

Note that an action determination system in accordance with the present disclosure is not limited to the one relating to the game as described in the embodiments above. The action determination system in accordance with the present disclosure may be used in order to, in another field, determine an action of an upper body of human or an animal and/or determine an action of a whole body, which is a combination of an action of an upper body and an action of a lower body of human or an animal. Examples of such a field include sport, care, and medicine (particularly, rehabilitation).

Note that the glove in accordance with the present disclosure is not limited to the boxing glove described in the embodiments above. The glove in accordance with the present disclosure can be any one that can be worn on a hand of a user and that is made from fiber and/or leather. Further, the glove in accordance with the present disclosure may be configured such that part of the hand of the user Us is externally exposed.

In the embodiments above, the position of the pressure sensor 13 disposed in the glove 10 is limited. However, the position of the pressure sensor 13 is not limited to this. In the glove 10, the pressure sensor 13 may be disposed at a position corresponding to a dorsal surface of the hand of a user or a position corresponding to a palm of a user. The position at which the pressure sensor 13 is disposed can be changed as appropriate depending on the intended use of the glove.

[Supplementary Note]

The present disclosure is not limited to the embodiments above, but can be altered by a skilled person in the art within the scope of the claims. The present disclosure also encompasses, in its technical scope, any embodiment derived by combining technical means disclosed in differing embodiments as appropriate.

### REFERENCE SIGNS LIST

1, 1A Action determination system
10 Glove
20 Armband
30 Controller
40 Force plate

1. A glove comprising a capacitive sensor, wherein:
conductive threads are woven into a fabric of the glove; and
the conductive threads serve as the capacitive sensor configured to detect, on a basis of a change in capacitance between the conductive threads adjacent to each other, a change in pressure applied to an area into which the conductive threads are woven.

2. The glove according to claim 1, wherein the conductive threads are woven into a position corresponding to a portion to which a pressure is to be applied in a hand of a user and which is in an area in contact with at least one selected from the group consisting of: a finger of the user, a palm of the user, and a dorsal surface of the hand of the user.

3. The glove according to claim 1, further comprising a transmitting section configured to transmit, to an external device, data indicating a change in pressure detected by the capacitive sensor,
the transmitting section being detachably attached to the glove.

4. An action determination system comprising:
the glove according to claim 1;
an acceleration sensor worn around an upper limb of a user and configured to detect a movement of the upper limb of the user; and
a controller configured to execute a determination process of determining an action of the upper limb of the user on a basis of a change in pressure detected by the capacitive sensor of the glove and a change in acceleration of the upper limb of the user detected by the acceleration sensor.

5. The action determination system according to claim 4, further comprising an external force measurement section configured to measure an external force applied from a lower limb of the user,
the controller being configured to further determine, in the determination process, an action of a whole body of the user on a basis of the action of the upper limb of the user determined and an external force measured by the external force measurement section.

6. The action determination system according to claim 5, wherein the external force measurement section is a force plate configured to measure a load applied from the user.

7. The action determination system according to claim 5, wherein the external force measurement section is footwear including a pressure sensor configured to measure a pressure applied from the user.

8. An action determination system comprising:
the glove according to claim 2;
an acceleration sensor worn around an upper limb of a user and configured to detect a movement of the upper limb of the user; and
a controller configured to execute a determination process of determining an action of the upper limb of the user on a basis of a change in pressure detected by the capacitive sensor of the glove and a change in acceleration of the upper limb of the user detected by the acceleration sensor.

9. The action determination system according to claim 8, further comprising an external force measurement section configured to measure an external force applied from a lower limb of the user,
the controller being configured to further determine, in the determination process, an action of a whole body of the user on a basis of the action of the upper limb of the user determined and an external force measured by the external force measurement section.

10. The action determination system according to claim 9, wherein the external force measurement section is a force plate configured to measure a load applied from the user.

11. The action determination system according to claim 9, wherein the external force measurement section is footwear including a pressure sensor configured to measure a pressure applied from the user.

12. An action determination system comprising:
the glove according to claim 3;
an acceleration sensor worn around an upper limb of a user and configured to detect a movement of the upper limb of the user; and
a controller configured to execute a determination process of determining an action of the upper limb of the user on a basis of a change in pressure detected by the capacitive sensor of the glove and a change in acceleration of the upper limb of the user detected by the acceleration sensor.

13. The action determination system according to claim 12, further comprising an external force measurement section configured to measure an external force applied from a lower limb of the user,
the controller being configured to further determine, in the determination process, an action of a whole body of the user on a basis of the action of the upper limb of the user determined and an external force measured by the external force measurement section.

14. The action determination system according to claim 13, wherein the external force measurement section is a force plate configured to measure a load applied from the user.

15. The action determination system according to claim 13, wherein the external force measurement section is footwear including a pressure sensor configured to measure a pressure applied from the user.
